(19) United States
(12) Patent Application Publication
(10) Pub. No.: US 2024/0324858 A1
(43) Pub. Date: Oct. 3, 2024

(54) ENDOSCOPE WITH NON-AXIAL CAMERA ORIENTATION

(71) Applicant: Blazejewski MEDI-TECH GmbH, Sexau (DE)

(72) Inventor: Reinhold BLAZEJEWSKI, Gutach (DE)

(21) Appl. No.: 18/613,204

(22) Filed: Mar. 22, 2024

(30) Foreign Application Priority Data
Mar. 29, 2023 (EP) 23165090.4

(51) Int. Cl. A61B 1/00; A61B 1/015; A61B 1/018; A61B 1/05; A61B 1/06

(52) U.S. Cl. CPC A61B 1/00179; A61B 1/00091; A61B 1/00096; A61B 1/015; A61B 1/018; A61B 1/05; A61B 1/0623; A61B 1/0676

(57) ABSTRACT

An endoscope includes a distal end surface having two surface sections that include an obtuse angle therebetween, wherein one surface section is oriented in an inclined manner relative to the longitudinal direction and the other surface section is transverse to the longitudinal direction. An image capturing device includes an objective lens having a light entrance window which is arranged in a flush mounted manner relative to the surface section. The image capturing device is arranged inside a cylindrical longitudinally oriented contour and has a viewing direction oriented obliquely to the longitudinal direction. The objective lens includes an optical axis having at least one bend, so that it includes a longitudinally oriented section and an obliquely oriented outer section. An illumination device is longitudinally oriented and includes an emission angle that is larger than an acceptance angle of the objective lens of the image capturing device.

### CROSS REFERENCE TO RELATED APPLICATION

This application claims the benefit of European Patent Application No. 23165090.4, filed Mar. 29, 2023, which is incorporated herein by reference in its entirety.

### TECHNICAL FIELD

The invention refers to an endoscope, particularly a spinal column endoscope or endoscope for other purposes, for example for urological applications.

### BACKGROUND

Endoscopes for inspection of operation sites are known from the prior art. They serve to allow the surgeon or another treating person a view on an inner body region of a patient.

WO 2022/043835 A1 and DE 10 2020 008 129 A1 disclose such an endoscope having a rod-shaped shank on the distal end of which a camera is arranged centrally. The camera comprises an illumination device that is formed by light-emitting diodes, which are arranged in a ring-shaped manner around the camera objective lens. The light-emitting diodes emit light through a ring-shaped window, wherein the emission directions of the individual light-emitting diodes are either parallel to the optical axis of the camera or directed away therefrom. The cones of light of the individual light-emitting diodes of the light-emitting collar overlap one another, so that in total a beam angle of the illumination is created, which can exceed 180°. However, the camera objective lens arranged centrally inside the light-emitting diode collar comprises an even larger acceptance angle.

DE 10 2014 208 754 A1 discloses an endoscope having a working channel with image capturing device and illumination device. The illumination device comprises multiple LED light sources, which are arranged in a quadrangle around the camera objective lens of the image capturing device. The viewing direction of the camera objective lens and the emission directions of the LED illuminations are identical and correspond to the longitudinal direction of the working channel.

Also CH 698 893 B1 discloses an endoscope with camera and illumination, which are commonly arranged on a distal end of an endoscope shank. Additional similar prior art is formed by WO 2019/158168 A1, WO 2021/175387, DE 10 2020 134 332 A1, U.S. Pat. No. 11,529,039 B2, WO 2021/175386 A1, US 2019/0246027 A1, EP 2 415 386 A1, DE 10 2013 201 809 A1, EP 2 311 366 B1 and U.S. Pat. No. 8,308,637 B2.

In the presented endoscopes it is principally assumed that the camera comprises an optical axis, which is orientated parallel to the longitudinal direction of the endoscope shank.

From the above-mentioned WO 2022/043835 A1 in addition an endoscope is known in which the unit consisting of an illumination and a camera is installed in oblique orientation relative to the longitudinal axis of the endoscope shank, so that the viewing direction of the camera is directed onto an instrument projecting from the working channel. In this arrangement, due to the instrument, remarkable shadow casting may result, which can result in impairment of visibility despite the large opening angles of the camera and the illumination device.

### SUMMARY

Starting therefrom it is one object of the invention to provide an endoscope with good visibility conditions.

This object is solved by means of an endoscope as described herein.

The endoscope according to the invention comprises a longitudinal shank, which extends along a longitudinal direction and comprises an image capturing device installed at a distal end as well as an illumination device, which is also installed at the distal end. The shank can be rigidly configured along its entire length. Alternatively, the shank can also be flexibly configured at one or more locations or entirely. In addition, the shank can comprise a distal end that can be specifically bent by means of respective actuation means.

The image capturing device comprises an image sensor and an objective lens. The illumination device comprises one or more LED light sources, preferably white light LEDs. On the distal end surface of the endoscope a first window assigned to the image capturing device as well as a second window assigned to the illumination device are provided. The first window and the second window are inclined to one another. This means that a surface normal of the first window and the surface normal of the second window include an acute angle with one another. If the windows are not planar but domed, the surface normal is in this regard to be positioned in the center of the window. The acute angle is opened in proximal direction, i.e. the point of intersection of the two surface normals is positioned distally away from the distal end surface of the endoscope. If the two surface normals do not intersect, the point of the closest approach of the two surface normals to one another is positioned distally in front of the distal end surface of the endoscope.

According to the inclination of the first window and the second window toward one another, also the two optical axes of the image capturing device and the illumination device include an acute angle. The illumination device comprises preferably an optical axis, which is orientated parallel to the longitudinal direction of the endoscope and thus parallel to an optionally provided working channel. The optical axis of the illumination device is the center axis of the cone of light emitted by the illumination device.

The optical axis of the image capturing device is, however, inclined relative to the longitudinal direction of the shank. In addition, the image capturing device is arranged offset in radial direction relative to the center axis of the shank. With this configuration a field of operation and the activity of a tool influencing biological tissue can be recognized particularly clear and well. Due to the longitudinal orientation of the illumination device, the emitted light comprises grazing light components in relation to a tool orientated parallel, i.e. some light rays of the emitted light beam extend substantially parallel to the tool extending from the working channel or parallel to the tool used in the endoscope. The grazing light components highlight image components and result in high-contrast images. The camera viewing onto the scene from the side overlooks the field of operation well and can particularly track axial movements of the tool well.

The first window and the second window can have planar light entrance surfaces respectively. They can be inserted in the distal end surface of the endoscope in a surface flush manner, whereby the endoscope can be cleaned and sterilized particularly well.

As mentioned, the image capturing device can comprise an optical axis, which is orientated in an acute angle being different from zero relative to the longitudinal direction. This particularly refers to the optical axis in its extension starting from the first window (light entrance window). Along the light path toward the image sensor, the optical axis can, however, also be subject to one or more direction changes and can be orientated parallel to the longitudinal direction inside the objective lens. This section of the optical axis is preferably arranged laterally offset relative to the center axis of the shank. This reduces the radial space requirement of the image capturing device compared to an entirely inclined positioned image capturing device having a continuously straight optical axis. This concept also allows the use of objective lenses having a remarkable extension in longitudinal direction. Such objective lenses can comprise multiple immovably or adjustably arranged lenses or other optical elements, such as prisms, mirrors or the like. High quality objective lenses can be used, which allow a high image quality. Thereby the endoscope can be configured particularly thin.

The optical axis of the illumination device forms, as already mentioned, a center axis of a light beam emitted by the illumination device. This cone of light comprises an illumination beam angle, which is preferably larger than the acceptance angle of the objective lens. Thereby the location of operation is spaciously and extensively illuminated.

It is advantageous, if the distal end surface of the endoscope comprises a first planar surface section, the surface normal of which is inclined relative to the longitudinal direction, wherein the distal end surface comprises a second planar surface section, the surface normal of which is orientated parallel to the longitudinal direction. Preferably the inclinations of the surface sections correspond to the inclinations of the windows provided therein. In doing so, the window surfaces can be inserted flush in surface sections of the distal end surface. The surface sections can transition into one another in a stepless manner. In a preferred embodiment, however, a (small) step is arranged between the two surface sections, in that the second surface section containing the second window is offset in proximal direction relative to the first surface section containing the first window. In doing so, a direct light irradiation from the second window into the first window and thus the image capturing device is avoided. This applies particularly, if the second window is completely clear, i.e. not configured in a manner providing a relevant light scattering.

In the simplest case, such an endoscope comprises no additional channel. However, the endoscope can also be provided with one or more working channels, which extend longitudinally through the shank. Such an endoscope cannot only be used for viewing a location of operation, but can also serve to influence it by means of a probe guided through the working channel to the location of operation. Preferably, the working channel comprises a mouth, which is entirely surrounded by the second surface section. In this case, the second surface section comprises at least one, preferably two light exit windows (second and third window). Between these windows of the illumination device and the window of the image capturing device additional channels can be provided, which serve, for example, for supply or discharging flushing fluid. The mouths of these channels can be arranged in a manner overlapping the two surface sections.

Preferably, the illumination device comprises only two light sources with two light exit windows that are arranged together on a virtual line transverse to the longitudinal direction, which is preferably approximately centrally positioned between the image capturing device and the working channel. In doing so, advantageous illumination and image capturing conditions result in a good overview of the operating area for the surgeon. In addition, an optimum use of space results, so that the endoscope can be configured particularly slim. In addition, the two light sources can be configured to emit light in different wavelength ranges. Additionally or alternatively, one, both or multiple light sources can comprise light emitting elements, for example LEDs, which are configured to emit light in different wavelength ranges. For example, one wavelength range can be in the range of visible light and can be, for example, white light. Another wavelength range can be in the range of invisible light, for example infrared, particularly near infrared light, or UV-light. The invisible light can serve for excitation of fluorescent markers.

The second and third window of the illumination device are thereby arranged at two different positions of the mouth of the working channel in direct proximity to the first surface section. With view from the image capturing device all light sources of the illumination device are positioned transverse within an angle of less than 180° in endoscopes having a working channel and preferably in an angle of less than 90° in endoscopes without working channel.

### BRIEF DESCRIPTION OF THE DRAWINGS

Additional details of advantageous embodiments of the endoscope are derived from embodiments and claims. In the following, embodiments of the invention are described in relation to the drawing. The drawing shows:

FIG. 1 an endoscope according to the invention in a schematic perspective illustration,

FIG. 2 the distal end of the endoscope according to FIG. 1 in perspective illustration,

FIG. 3 the distal end of the endoscope according to FIGS. 1 and 2 in longitudinal section,

FIG. 4 a front view of the distal end surface of the endoscope according to FIGS. 1 to 3,

FIG. 5 the distal end of a modified embodiment of the endoscope according to the invention in perspective illustration,

FIG. 6 the distal end of the endoscope according to FIG. 5 in longitudinal section and

FIG. 7 a view of the distal end surface of the endoscope according to FIGS. 5 and 6.

### DETAILED DESCRIPTION

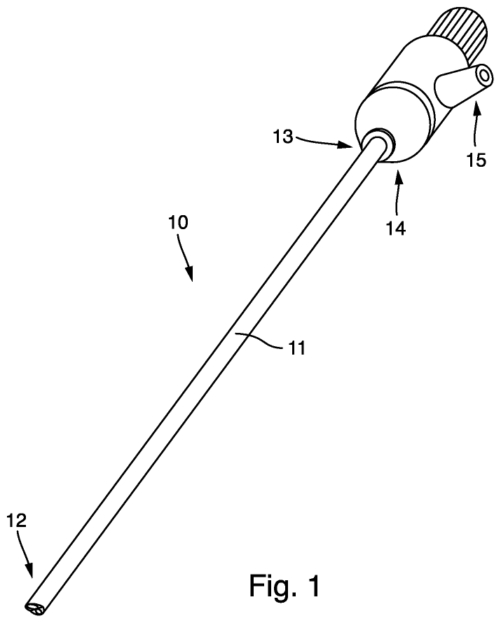

In FIG. 1 an endoscope 10 is illustrated that serves for endoscopic treatment of patients and can be used, for example, as spinal column endoscope or also as endoscope for other purposes. The endoscope 10 comprises a longitudinal shank 11, which can be configured as rigid shank or in another variant also as flexible shank. It extends from its distal end 12 to its proximal end 13 where it is held on an endoscope head 14. On the endoscope head 14 a connection 15 can be provided that can be configured, for example, for connecting a supply line, for example for supplying electrical energy, or also as connection for inserting a probe or as connection for supplying or discharging of fluids. In addition, a connection for signal transmission can be provided on the endoscope head, e.g. for image signal transmission.

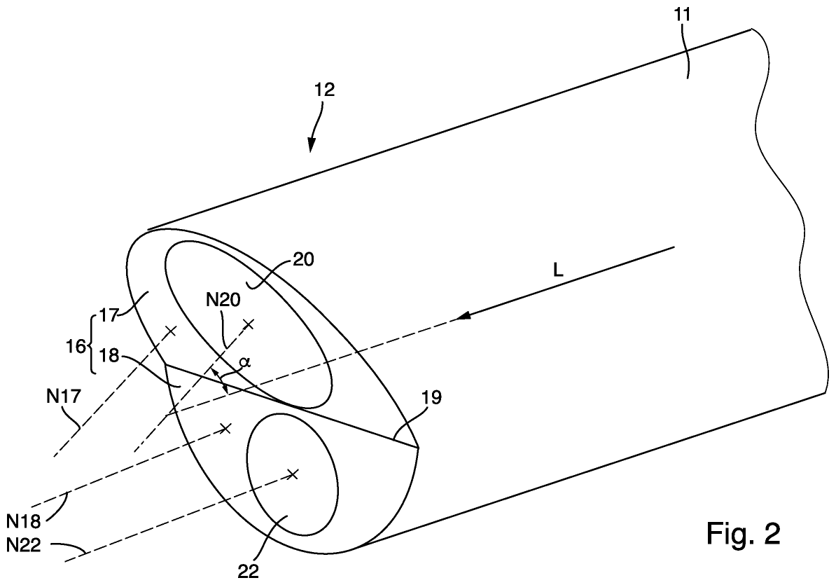

The distal end 12 of endoscope 10 is individually illustrated in FIG. 2. The longitudinal shank 11 that is preferably circular in cross-section defines a longitudinal direction L by its longitudinal extension, which is symbolized in FIG. 2 by means of an arrow.

The longitudinal cylindrical shank 11 comprises a distal end surface 16 at its distal end 12 having a preferably planar first surface section 17 and a preferably also planar second surface section 18, which transition into one another at a preferably straight separation line 19. The surface sections 17, 18 can transition steplessly into one another. Alternatively, at the separation line 19 also a step can be provided, as apparent from FIG. 3.

In the first surface section 17 a first window 20 is arranged, which serves as light entrance window of an image capturing device 21 apparent from FIG. 3. In the second surface section 18 a second window 22 is arranged that serves as light exit window of an illumination device 23. The first window 20 and the second window 22 can comprise distally a planar surface and can end flush with the respective surface section 17, 18 entirely surrounding them.

The surface section 17 comprises a surface normal N17 that is inclined in an acute angle w relative to the longitudinal direction L. Starting from the separation line 19, the surface section 17 extends increasingly in distal direction. On the contrary, the surface section 18 comprises a surface normal N18, which is orientated parallel to the longitudinal direction L. The surface normals N17, N18 thus intersect distally from the distal end surface 16 in a point P. Also, distally in front of the distal end surface 16 the surface normals N20, N22 of first and second windows 20, 22 intersect.

As illustrated in FIG. 3, the surface normal N20 coincides with the optical axis of the image capturing device 21 extending in front of the distal end 12. The image capturing device 21 comprises an objective lens 24 and an image capturing sensor 25. The optical path of the objective lens can comprise lenses and other optical elements, such as mirrors or prisms, so that the optical axis inside the objective lens is subject to at least one deflection and comprises a section 26 extending parallel to the longitudinal direction L there. By means of at least one or also multiple bends in the optical axis it is achieved that the entire objective lens 24 and the image capturing sensor 25 can be located inside a cylindrical contour orientated in longitudinal direction L, although the objective lens 24 and thus the entire image capturing device 21 comprises a viewing direction, represented by surface normal N20, extending under an angle a obliquely to the longitudinal direction L.

The objective lens 24 comprises preferably an acceptance angle o smaller than 120°, preferably of at most 80° or less (inside air). Under water the acceptance angle o can have an amount of approximately 65°.

The illumination device 23 comprises a light-emitting semi-conductor element, such as an LED 27, which can be configured as white light LED. Preferably, it is a short wavelength light emitting LED having luminescent material. Alternatively, also multiple different colored LED-chips can be provided, the light of which can be superimposed to white light or light of adjustable color. The light emitted by LED 27 passes second window 22, which is preferably entirely transparent or translucent. The light exit angle marking the beam angle b of a light cone exiting the window 22 is preferably larger than the acceptance angle o of the objective lens 24. The emission or light exit angle can be between 90° and 120°, for example. The center axis of the light cone emitted from the illumination device 23 is marked by surface normal N22 orientated parallel to the longitudinal direction L.

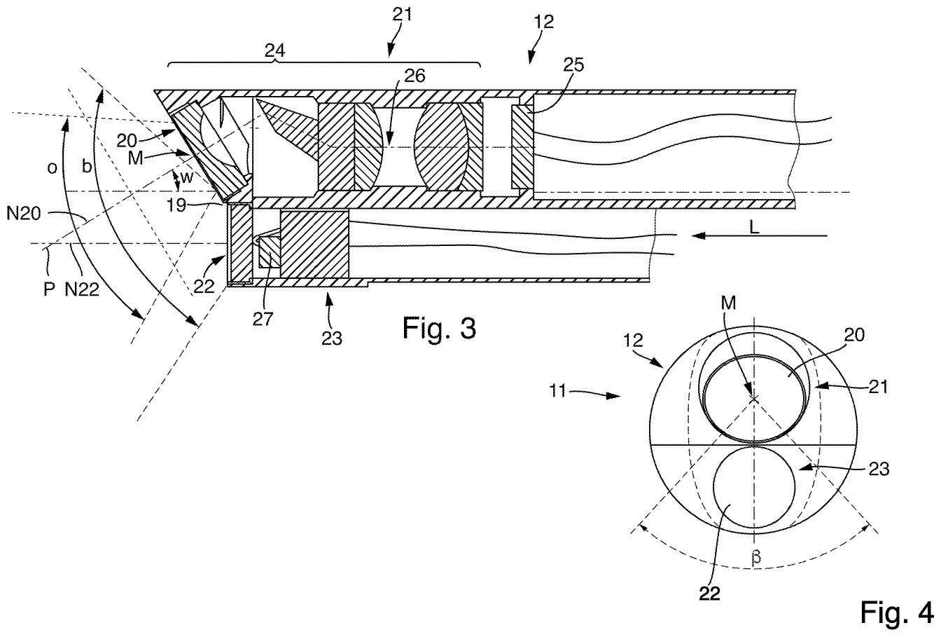

As illustrated in FIG. 4, the illumination device 23 is arranged only on one side of image capturing device 21. With view from a center point M of first window 20 the illumination device 23 as well as the light exit window 22 thereof is positioned inside an angle B illustrated by dashed lines in FIG. 4, which is preferably less than 90°. Due to this arrangement, a particularly slim shank 11 can be designed. It can comprise a circular cross-section, as illustrated in FIG. 4. Alternatively, shank 11 can also be overly configured, as indicated by the dashed lines.

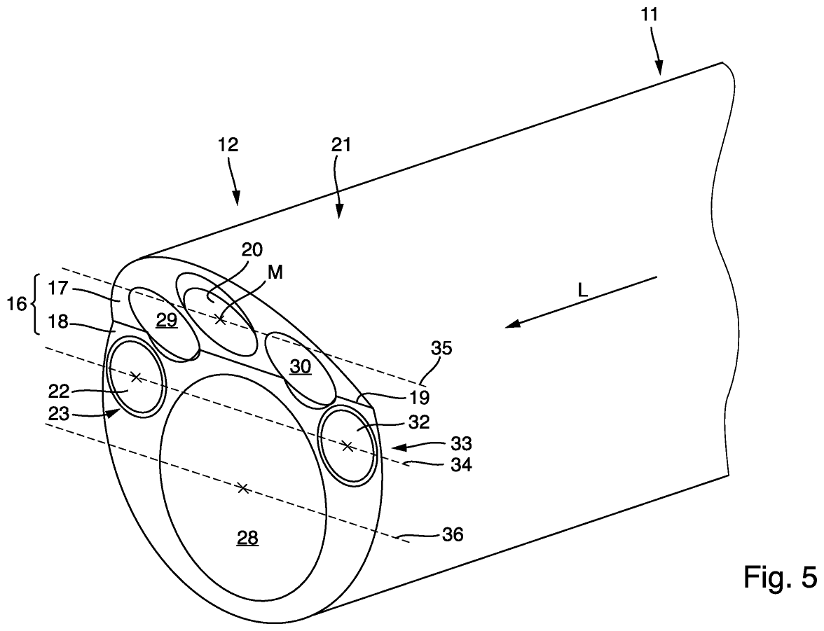

While FIGS. 1 to 4 illustrate an endoscope without working channel, an endoscope having a working channel 28 as well as optionally additional channels, e.g. flushing channels 29, 30, is illustrated in FIGS. 5 to 7. The working channel 28 as well as the flushing channels 29, 30 preferably extend in longitudinal direction L through the entire shank 11 from its distal end 12 up to its proximal end 13 and toward the endoscope head 14 where they are connected with respective connections.

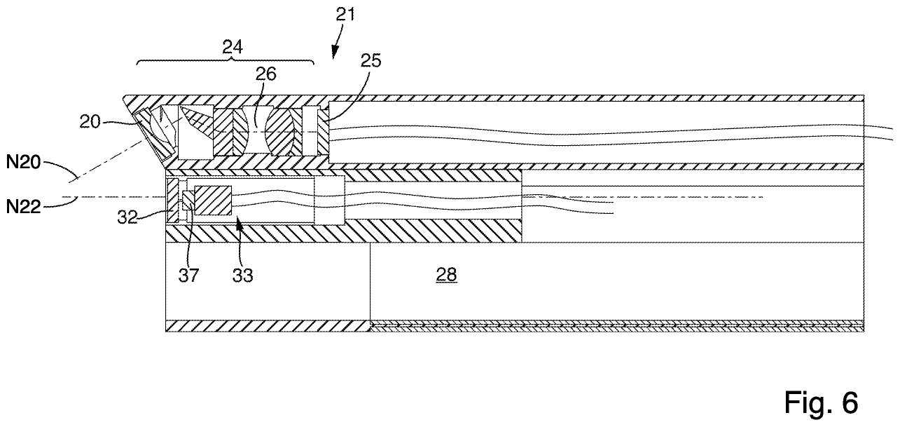

The endoscope according to FIG. 5 also comprises an image capturing device 21, as described above, the light entrance window of which is the first window 20. The description provided with reference to FIGS. 1 to 4 of endoscope 10, particularly of the image capturing device 21, the objective lens 24 and the image sensor 25 thereof as well as shank 11 and the distal end surface 16 applies accordingly for the image capturing device 21 illustrated in FIG. 6 on the basis of the already introduced reference signs. The distal end surface 16 is again divided into surface sections 17, 18, which are orientated relative to the longitudinal direction L and adjoin one another at the step or separation line 19, as described in the context of FIGS. 1 to 4.

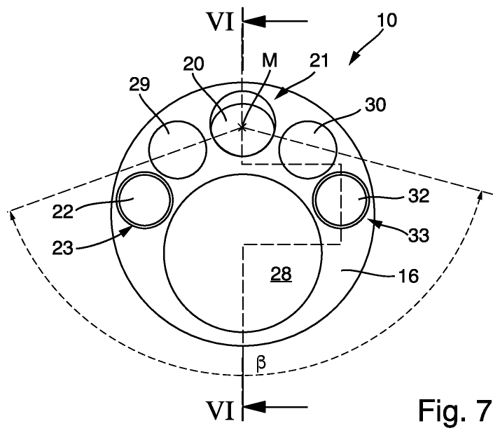

As apparent from FIGS. 5 and 7, however, in addition to the second window 22, a third window 32 is present that serves as light exit window and is part of an additional illumination device 33. The illumination device 33 is configured identically to the illumination device 23. The illumination devices 23, 33 are symmetrically arranged relative to a plane that extends in longitudinal direction L and centrally through working channel 28 and light entrance window 20. As shown in FIG. 5, the light exit windows 22, 32 are thereby arranged with their center points on a line 34, which is arranged between parallels 35, 36 arranged parallel to the separation line 19, wherein the parallel 35 extends centrally through the first window 20 and the parallel 36 extends centrally through the working channel 28. Preferably the line 34 is approximately centered between lines 35, 36. The flushing channel 29 is preferably arranged between the first window 20 and the second window 22. The mouth of flushing channel 30 is preferably arranged between first window 20 and third window 32. In doing so an optimum use of space in shank 11 results. Vice versa thereby particularly slim endoscopes can be designed.

The configuration of illumination device 33 corresponding to that of illumination device 23 is apparent from FIG. 6, which is a sectional illustration according to sectional line VI-VI of FIG. 7. The description of the illumination device 23 applies accordingly for the illumination device 33 and its LED 37. As apparent from FIG. 7, all illumination devices 23, 33 of endoscope 10 according to FIG. 7 are arranged inside an angle β, the vertex of which is marked by the center point M of first window 20, wherein this angle β is smaller than 180°.

The endoscope 10 described so far operates as follows:

For the use on the patient the endoscope 10 is inserted through a suitable opening of the body of the patient in an area thereof that is to be optically inspected. In case of an endoscope according to FIGS. 1 to 4, the illumination device 23 is activated. The image captured by image sensor 25 is transmitted in form of image signals to a suitable monitor. The monitor can be a display provided on the endoscope head 15, an individual display separated therefrom or also VR-glasses of the treating person. If an instrument is used, which is brought into the field of view of the image capturing device 21 in another manner, it can be seen as well and its use can be supervised and controlled. Thereby the endoscope 10 is also suitable for instruments, which are inserted parallel to the shank thereof into the patient and used. Due to the special arrangement of the illumination of the illumination device orientated in longitudinal direction L and the viewing direction of the image capturing device 21 inclined toward the longitudinal direction L, a good overview of the field of operation and the operation of the instrument is guaranteed.

This applies particularly to the endoscope according to FIGS. 5 to 7 in which the working direction of a probe inserted through the working channel 28 is defined by means of the working channel 28 parallel to the longitudinal direction L. Due to the arrangement of two illumination devices 23, 33 on both sides of working channel 28, however slightly offset toward the image capturing device 21, a good illumination of the field of operation without shadow casting and with optimized viewing direction for the operation of the instrument is achieved.

The endoscope 10 according to the invention comprises a distal end surface 16 having two surface sections 17, 18 that include an obtuse angle with one another, wherein one surface section 17 is orientated in an inclined manner relative to the longitudinal direction, whereas the other surface section 18 is positioned transverse to the longitudinal direction L. The image capturing device 21 comprises an objective lens having a light entrance window 20, which is preferably arranged in a flush mounted manner relative to the surface section 17, wherein the image capturing device 21 is arranged inside a cylindrical longitudinally orientated contour and nevertheless comprises a viewing direction orientated obliquely to the longitudinal direction. The objective lens 24 being part of the image capturing device 21 comprises an optical axis having at least one bend, so that it comprises a longitudinally orientated section 26 and an obliquely orientated outer section N20. On the contrary, the illumination device 23 (33) is longitudinally orientated and comprises an emission angle being larger than the acceptance angle o of the objective lens 24 of the image capturing device 21.

### LIST OF REFERENCE SIGNS

10 endoscope
11 shank
12 distal end of shank 11
13 proximal end of shank 11
14 endoscope head
15 connection on endoscope head
L longitudinal direction
16 distal end surface
17 first surface section of distal end surface 16
18 second surface section of distal end surface 16
19 separation line
20 first window/light entrance window
21 image capturing device
22 second window/light exit window
23 illumination device
b beam angle of der illumination device 23
N17 surface normal of surface section 17
N18 surface normal of surface section 18
N20 surface normal of first window 20
N22 surface normal of second window 22
P intersection point, distal to end surface 16
24 objective lens
o acceptance angle of objective lens 24
25 image sensor
26 section of optical axis of objective lens 24
27 LED
M center point of first window 20
α, β angle
28 working channel
29, 30 flushing channel
32 third window/light entrance window
33 third illumination device
34 line
35 parallel
36 parallel
37 LED

1. An endoscope (10), comprising:
a longitudinal shank (11), which extends along a longitudinal direction (L) and including a distal end surface (16) in which a first window (20) and a second window (22) are arranged, whereby an image capturing device (21) is assigned to the first window (20) and an illumination device (23) is assigned to the second window (22),
wherein the first window (20) and the second window (22) are inclined toward one another.

2. The endoscope according to claim 1, wherein the first window (20) comprises a planar light entrance surface.

3. The endoscope according to claim 1, wherein the second window (22) comprises a planar light exit surface.

4. The endoscope according to claim 1, wherein the illumination device (23) comprises an optical light exit axis (N22), which is orientated parallel to the longitudinal direction (L) and that the image capturing device (21) comprises an optical axis (N20) that is orientated in an acute angle (a) different from zero relative to the longitudinal direction (L).

5. The endoscope according to claim 1, wherein the image capturing device (21) comprises a camera objective lens (24) and an image sensor (25), which are both immovably installed in a distal end (12) of the shank (11) and that the illumination device (23) comprises an LED-light source 27, which is immovably installed in the distal end (12) of the shank (11).

6. The endoscope according to claim 5, wherein the camera objective lens (24) comprises an objective acceptance angle (o), and the illumination device (23) comprises an illumination beam angle (b), which is larger than the objective acceptance angle (o).

7. The endoscope according to claim 1, wherein the distal end surface (16) comprises a first surface section (17), a surface normal (N17) of which is inclined relative to the longitudinal direction (L), wherein the first window (20) is arranged completely inside the first surface section (17) and wherein the distal end surface (16) comprises a second surface section (18), a surface normal (N18) of which is oriented parallel to the longitudinal direction (L), wherein the second window (22) is entirely arranged inside the second surface section (18).

8. The endoscope according to claim 1, further comprising a working channel (28) that extends longitudinally through the shank (11).

9. The endoscope according to claim 7, further comprising a working channel (28) that extends longitudinally through the shank (11), wherein the working channel (28) comprises a mouth that is entirely surrounded by the second surface section (18).

10. The endoscope according to claim 1, further comprising a third window (32) arranged in the distal end surface (16) and to which an illumination device (33) is assigned.

11. The endoscope according to claim 9, further comprising a third window (32) arranged in the distal end surface (16) and to which an illumination device (33) is assigned, wherein the second and the third window (22, 32) are arranged at two different sides of the mouth of the working channel (28) in direct proximity to the first surface section (17).

12. The endoscope according to claim 1, wherein the illumination device (23) is arranged in relation to the image capturing device (21) exclusively laterally within an angle, which is smaller than 180°.

13. The endoscope according to claim 8, wherein at least one additional channel (29) is provided parallel to the working channel (28) inside the shank (11), wherein a diameter of the additional channel (29) is less than an inner diameter of the working channel (28).

14. The endoscope according to claim 13, wherein the at least one additional channel (29) is arranged between the first window (20) and the second window (22).

15. The endoscope according to claim 13, wherein the distal end surface (16) comprises a first surface section (17), a surface normal (N17) of which is inclined relative to the longitudinal direction (L), wherein the first window (20) is arranged completely inside the first surface section (17) and wherein the distal end surface (16) comprises a second surface section (18), a surface normal (N18) of which is oriented parallel to the longitudinal direction (L), wherein the second window (22) is entirely arranged inside the second surface section (18), wherein the at least one additional channel (29) comprises a mouth which is arranged inside the first surface section (17) as well as inside the second surface section (18).
